(19) United States
(12) Patent Application Publication
(10) Pub. No.: US 2020/0194097 A1
(43) Pub. Date: Jun. 18, 2020

(54) METHOD FOR IDENTIFYING PLANT IncRNA AND GENE INTERACTION

(71) Applicant: Beijing Forestry University, Beijing (CN)

(72) Inventors: Deqiang Zhang, Beijing (CN); Mingyang Quan, Beijing (CN); Qingzhang Du, Beijing (CN); Liang Xiao, Beijing (CN); Wenjie Lu, Beijing (CN)

(21) Appl. No.: 16/579,916

(22) Filed: Sep. 24, 2019

(30) Foreign Application Priority Data
Dec. 18, 2018 (CN) 201811549079.8

(51) Int. Cl. G16B 20/20; G16B 30/10; C12Q 1/6869; G16B 20/40

(52) U.S. Cl. CPC G16B 20/20; G16B 20/40; C12Q 1/6869; G16B 30/10

(57) ABSTRACT

A method for identifying plant lncRNA and gene interaction includes obtaining population SNP genotype data of the lncRNA and the gene; obtaining population expression abundance data of the gene in the studied tissue; and obtaining target trait population phenotypic data. When three restrictive conditions defined by the method are satisfied at the same time, it is indicated that the lncRNA and the gene are interacted with each other and together affect the phenotypic variation of the target trait of the plant. The method is used to accurately detect the interaction relationship between P. tomentosa lncRNA LNC-0052611 and gene Pto-COMT25, and the interaction relationship affects the phenotypic variation of a diameter at breast height of P. tomentosa.

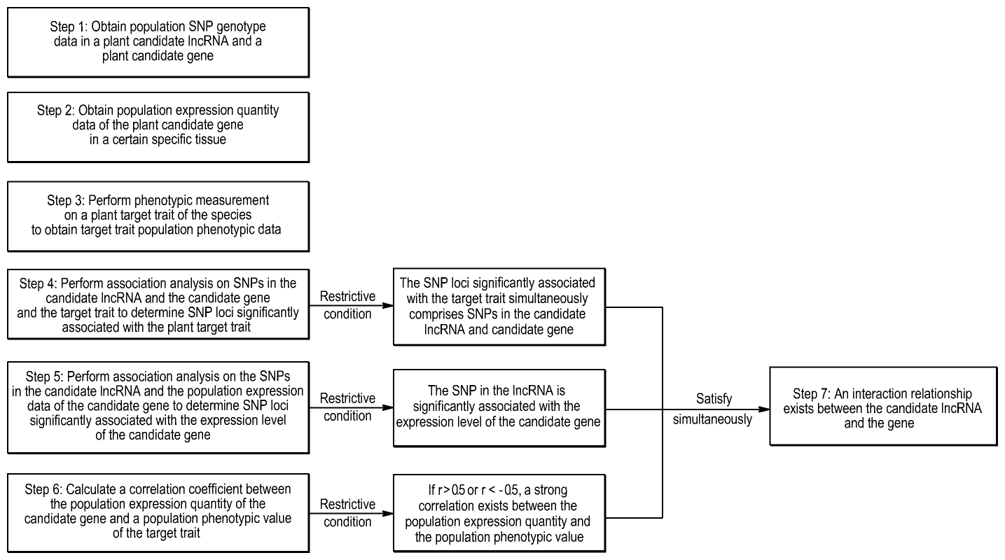

### CROSS-REFERENCE TO RELATED APPLICATION

This application claims priority to Chinese application number 201811549079.8, filed Dec. 18, 2018. The above-mentioned patent application is incorporated herein by reference in its entirety.

### TECHNICAL FIELD

The present invention relates to the field of molecular genetics techniques, and in particular, to a method for identifying plant lncRNA.

### BACKGROUND

Long non-coding RNA (“lncRNA”) refers to a class of regulatory transcripts that have no protein-coding function and are greater than 200 nt in length. Researches indicate that the lncRNA can regulate the expression of genes at multiple levels, thus affecting the growth and development of plants, such as rice pollen fertility and Arabidopsis photomorphogensis. Plant growth is a complex process, which is regulated by multiple genes at multi-level, and the interactions between various genetic factors are more diverse. At present, the mechanisms of action of the lncRNA are still unclear. The study about the interaction between the lncRNA and the gene is mainly based on the principle of complementary base pairing, and the lncRNA could regulate the gene expression by interacting with its target gene in cis or trans at transcriptional, post-transcriptional, and epigenetic level.

At present, the interactions between the plant lncRNAs and their target genes only consider the sequence similarity between two transcripts, which would cause the false positive results for identification of the interactions between lncRNA and target gene. Moreover, the prediction mode is relatively simple and cannot accurately detect a functional gene that is interacted with the lncRNA. Therefore, the prior art lacks a method for accurately identification of plant lncRNA and target gene interaction.

Thus, it is desirable to provide a method for identification of plant lncRNA and target gene interaction, to address these and other deficiencies of the current art.

### SUMMARY

To achieve the above purposes and overcome the technical defects in the art, the method is provided that can accurately identify the interaction relationship between a plant lncRNA and a gene.

In one embodiment, a method for identifying plant lncRNA and target gene interaction includes the following steps: (1) obtaining population SNP genotype data of a plant candidate lncRNA and a plant candidate gene; (2) obtaining population expression abundance data of the plant candidate gene in the tested tissue; (3) performing phenotypic measurement on a tested trait to obtain the population phenotypic data; (4) performing association analysis using the population SNP genotype data in step (1) and the target trait population phenotypic data in step (3) to determine SNP loci significantly associated with the plant target trait; the determining condition including: the SNP loci significantly associated with the plant target trait simultaneously include the SNP loci in the plant candidate lncRNA and the SNP loci in the plant candidate gene; (5) performing association mapping analysis using the population SNP genotype data in step (1) and the population expression quantity data in step (2) to determine the SNP loci significantly associated with the expression level of the plant candidate gene; the determining condition including: the SNP loci within the plant candidate lncRNA are significantly associated with the expression level of the candidate gene; (6) calculating the correlation coefficient r between the population expression level data in step (2) and the target trait population phenotypic data in step (3) to determine the correlation therebetween; the determining condition including: the correlation coefficient r>0.5 or r<−0.5; the formula for calculating the correlation coefficient r being as follows:

$$\frac{N \sum XY - \sum X \sum Y}{\sqrt{N \sum {X}^{2} - {( \sum X )}^{2}} \sqrt{N \sum {Y}^{2} - {( \sum Y )}^{2}}}$$

where X is the expression quantity data of the plant candidate gene in the detected tissue, and Y is the target trait population phenotypic data; and (7) when the determining conditions in steps (4) through (6) are satisfied simultaneously, indicating that the plant candidate lncRNA and the plant candidate gene have an interaction relationship, and together affect the phenotypic variation of the plant target trait.

In one embodiment, the plant candidate lncRNA and the plant candidate gene in step (1) are expressed in the same tissue of a plant.

In another embodiment, the population SNP genotype data in step (1) is obtained based on plant whole genome re-sequencing data.

In a further embodiment, the frequency of the population SNP genotype of the plant candidate lncRNA and the plant candidate gene in step (1) is greater than 10%.

In yet another embodiment, software used for the association analysis in step (4) and step (5) is TASSEL v5.0.

In one embodiment, a model used for the association analysis is a mixed linear model.

In another embodiment, the association mapping method includes: obtaining a significance level P value of each SNP locus associated with the phenotype by using the software TASSEL v5.0; performing FDR test on the P value by using Q-value software to obtain a Q value; and screening SNP loci with P≤0.01 and Q≤0.1 as SNP loci significantly associated with the plant target trait.

In a further embodiment, the method for obtaining the population SNP genotype data in step (1) includes: performing whole genome sequencing on each individual in the used natural population to respectively obtain genomic sequences; performing sequence alignment on the genomic sequences to obtain whole genome genotype SNP data; and performing alignment using the plant candidate lncRNA and the plant candidate gene to the reference genome, and combining the whole genome genotype SNP data to obtain the population SNP genotype data of the plant candidate lncRNA and the plant candidate gene.

Embodiments of the invention provide a method for identifying plant lncRNA and gene interaction. The previous interaction relationship between the lncRNA and the target gene only considers the sequence similarity, and the identified gene interacted with the lncRNA has false positive. Moreover, identifying the interaction relationship between the lncRNA and the gene through sequence similarity lacks a biological significance. Therefore, the present invention utilizes a population genetics strategy to provide a method for identifying plant lncRNA and target gene interaction, and can accurately detect a functional gene interacted with the lncRNA, which has important biological significance.

The results of examples of the present invention show that the interaction relationship between the Populus tomentosa lncRNA LNC-0052611 and the gene Pto-COMT25 is obtained by the method provided by the present invention, and the interaction relationship affects the phenotypic variation of a Diameter at Breast Height (DBH) of the P. tomentosa.

### BRIEF DESCRIPTION OF THE DRAWING

Various additional features and advantages of the invention will become more apparent to those of ordinary skill in the art upon review of the following detailed description of one or more illustrative embodiments taken in conjunction with the accompanying drawing. The accompanying drawing, which is incorporated in and constitutes a part of this specification, illustrates one or more embodiments of the invention and, together with the general description given above and the detailed description given below, explain the one or more embodiments of the invention

The sole FIGURE is a flowchart showing the analysis of an identification method according to one embodiment of the invention.

### DETAILED DESCRIPTION

The following clearly and completely describes the technical solutions in the embodiments of the present invention with reference to the accompanying drawings in the embodiments of the present invention. To make objectives, features, and advantages of the present invention clearer, the following describes embodiments of the present invention in more detail with reference to the accompanying drawing and specific implementations.

In some embodiments, the present invention provides a method for identifying plant lncRNA and gene interaction, including the following steps: (1) obtaining population SNP genotype data of a plant candidate lncRNA and a plant candidate gene; (2) obtaining population expression data of the plant candidate gene in the studied tissue; (3) performing phenotypic measurement of a tested trait to obtain the population phenotypic data; (4) performing association analysis using the population SNP genotype data in step (1) and the target trait population phenotypic data in step (3) to determine SNP loci significantly associated with the plant target trait; the determining condition including: the SNP loci significantly associated with the plant target trait simultaneously include the SNP loci in the plant candidate lncRNA and the SNP loci in the plant candidate gene; (5) performing association analysis using the population SNP genotype data in step (1) and the population expression quantity data in step (2) to determine the SNP loci associated with the expression level of the plant candidate gene; the determining condition including: the SNP loci within the plant candidate lncRNA are significantly associated with the expression level of the candidate gene; (6) calculating the correlation coefficient r between the population expression level data in step (2) and the target trait population phenotypic data in step (3) to determine the correlation therebetween; the determining condition including: the correlation coefficient r>0.5 or r<−0.5; the formula for calculating the correlation coefficient r being as follows:

$$\frac{N \sum XY - \sum X \sum Y}{\sqrt{N \sum {X}^{2} - {( \sum X )}^{2}} \sqrt{N \sum {Y}^{2} - {( \sum Y )}^{2}}}$$

where X is the expression quantity data of the plant candidate gene in the detected tissue, and Y is the target trait population phenotypic data; and (7) when the determining conditions in steps (4) through (6) are satisfied simultaneously, indicating that the plant candidate lncRNA and the plant candidate gene have an interaction relationship, and together affect the phenotypic variation of the plant target trait.

The method obtains the population SNP genotype data of the plant candidate lncRNA and the plant candidate gene.

The type of the plant is not particularly limited in the present invention, and in examples of the present invention, the plant is preferably P. tomentosa.

In one embodiment, the plant candidate lncRNA and the plant candidate gene are preferably expressed in the same tissue of the plant. In another embodiment, the frequency of the population SNP genotype of the plant candidate lncRNA and the plant candidate gene is preferably greater than 10%.

In a further embodiment, the population SNP genotype data is preferably obtained based on plant whole genome re-sequencing data. The method for obtaining the population SNP genotype data preferably includes: performing whole genome sequencing on each individual in the used natural population to respectively obtain genome sequences; performing sequence alignment on the genome sequences to obtain the whole genome genotype SNP data; and performing alignment using the plant candidate lncRNA and the plant candidate gene to the reference genome, and combining the whole genome genotype SNP data to obtain the population SNP genotype data of the plant candidate lncRNA and the plant candidate gene. The software used for the alignment is preferably Bioedit. The reference gene is preferably a published genome of the plant. The method first begins with whole genome re-sequencing, where each SNP locus on the genome has a fixed position on the genome. Secondly, the positions of the two candidate genes (the lncRNA and the candidate gene) in the reference genome can be determined by sequence alignment. Therefore, SNP data in the candidate gene can be determined based on the positions of the candidate genes in the genome.

In some embodiments, whole genome sequencing is preferably respectively performed on individuals in the used natural population to respectively obtain genomic sequences. The method for sequencing the whole genome is not particularly limited in the present invention, and a conventional sequencing method can be used.

In a further embodiment, sequence alignment is performed on the genomic sequences to obtain whole genome SNP genotype data. The method for sequence alignment is not particularly limited in the present invention, and a conventional sequence alignment method can be used.

In yet another embodiment, alignment is performed using the plant candidate lncRNA and the plant candidate gene to a reference genome, and the whole genome genotype SNP data is combined to obtain the population SNP genotype data.

In one embodiment, population expression quantity data of the plant candidate gene in the tissue is obtained. The method for obtaining the population expression quantity data of the plant candidate gene in the tissue is not particularly limited in the present invention, and a conventional method for obtaining the expression quantity data of the tissue can be used. The tissue is preferably a certain particular tissue. The tissue expressed by the plant candidate gene in the population is preferably identical to the tissue expressed by the plant candidate lncRNA and the plant candidate gene. The tissue is not particularly limited in the present invention, and any tissue of the plant can be used.

In another embodiment, phenotypic measurement is performed on a plant target trait to obtain the population phenotypic data. The method for performing phenotypic measurement on the plant target trait is not particularly limited in the present invention, and a conventional method can be used. The target trait is not particularly limited in the present invention, and any trait of the plant can be used.

In a further embodiment, association analysis is performed using the population SNP genotype data and the target trait population phenotypic data to determine an SNP locus significantly associated with the plant target trait, where the determining condition includes: the SNP loci significantly associated with the plant target trait simultaneously include SNP loci in the plant candidate lncRNA and SNP loci in the plant candidate gene. Software used for the association analysis is preferably TASSEL v5.0. A model used for the association analysis is preferably a mixed linear model. The method of association analysis preferably includes: obtaining a significance level P value of each SNP locus associated with phenotype by using software TASSEL v5.0; performing FDR test on the P value by using Q-value software to obtain a Q value; and screening SNP loci with P≤0.01 and Q≤0.1 as SNP loci significantly associated with the plant target traits. The purpose of performing multiplex test to obtain a Q value is to exclude false positive results. The resulting significantly associated SNP loci need to contain SNP loci both from the plant candidate lncRNA and gene, but the number and attributes of the SNP loci are not limited.

In a further embodiment, association analysis is performed on the population SNP genotype data and the population expression data to determine the SNP loci associated with the expression level of the plant candidate gene, where the determining condition includes: the SNP loci of the plant candidate lncRNA is significantly associated with the expression level of the candidate gene. The method for performing association analysis on the population SNP genotype data and the population expression data is the same as the method for performing association analysis on the population SNP genotype data and the target trait population phenotypic data, and will not be described herein. The SNP loci in the plant candidate lncRNA need to be significantly associated with the expression level of the plant candidate gene, but the number and attributes of the SNP loci are not limited.

In yet another embodiment, the correlation coefficient r between the population expression data and the target trait population phenotypic data is calculated to determine the correlation therebetween, where the determining condition includes: the correlation coefficient r>0.5 or r<−0.5, and the formula for calculating the correlation coefficient r is as follows:

$$\frac{N \sum XY - \sum X \sum Y}{\sqrt{N \sum {X}^{2} - {( \sum X )}^{2}} \sqrt{N \sum {Y}^{2} - {( \sum Y )}^{2}}}$$

where X is the expression quantity data of the plant candidate gene in the detected tissue, and Y is the target trait population phenotypic data.

In one embodiment, if the correlation coefficient r>0.5 or r<−0.5, a strong correlation exists between the population expression data and the target trait population phenotypic data, indicating that the expression level of the plant candidate gene can greatly affect the variation of the target trait. The correlation coefficient r value ranges from −0.5 to 0.5, indicating that the correlation therebetween is low.

In another embodiment, when the determining conditions in steps (4) through (6) are satisfied simultaneously, it is indicated that the plant candidate lncRNA and the plant candidate gene have an interaction relationship, and together affect the phenotypic variation of the plant target trait. In the present invention, the interaction pre-selection between the plant candidate lncRNA and the plant candidate gene is premised on the regulation of the selected target trait.

The method for identifying plant lncRNA and gene interaction according to the present invention will be further described in detail below with reference to specific examples. The technical solutions of the present invention include, but are not limited to, the following examples.

### Example 1

The interaction between the P. tomentosa lncRNA LNC-0052611 and the gene Pto-COMT25 is identified using a method for identifying plant lncRNA and gene interaction provided by embodiments of the present invention.

Step S1: SNP genotype data of the lncRNA LNC-0052611 and the gene Pto-COMT25 in the natural population of P. tomentosa is obtained, including the following specific steps:

Step S11: the one-year-old “LM50” clone of P. tomentosa planted in Guan County, Shandong Province is taken as experimental material, the mature xylem was collected for transcriptome sequencing, and in order to prevent RNA degradation, the collected mature xylem was placed in a liquid nitrogen environment (−196° C.) for storage immediately after the collection. RNA of the collected mature xylem was extracted using a Plant Qiagen RNAeasy kit (Qiagen China, Shanghai, China), and is transferred to a biotechnology company for lncRNA and transcriptome sequencing after quality assessment to detect lncRNA and mRNA expressed in the tissue. The lncRNA LNC-0052611 and the gene Pto-COMT25 expressed in the tissue are selected as candidate genetic factors, and the interaction relationship therebetween is further analyzed.

Step S12: Firstly, the genomic DNA is extracted from the 435 individuals of the natural population of P. tomentosa, which is used for re-sequencing, and the poplar reference genome, i.e. the genome of P. trichocarpa, is used for sequence alignment to obtain whole genome SNP genotype data. Secondly, the P. tomentosa lncRNA LNC-0052611 and the gene Pto-COMT25 were aligned to reference genome using bioedit software in order to extract population SNP genotype data of the two candidate genetic factors. Finally, the loci with the SNP genotype frequencies greater than 10% are screened as candidate SNPs for P. tomentosa lncRNA LNC-0052611 and the gene Pto-COMT25. See Table 1 for details of candidate SNPs.

TABLE 1
SNP information in LncRNA LNC-0052611 and gene Pto-COMT25

| Gene name | SNP position | SNP name | SNP genotype |
|---|---|---|---|
| LNC-0052611 | LncRNA | SNP1 | C/T |
| LNC-0052611 | LncRNA | SNP2 | A/T |
| LNC-0052611 | LncRNA | SNP3 | C/T |
| LNC-0052611 | LncRNA | SNP4 | A/G |
| LNC-0052611 | LncRNA | SNP5 | A/G |
| LNC-0052611 | LncRNA | SNP6 | A/G |
| LNC-0052611 | LncRNA | SNP8 | C/T |
| LNC-0052611 | LncRNA | SNP9 | C/T |
| LNC-0052611 | LncRNA | SNP10 | C/G |
| LNC-0052611 | LncRNA | SNP11 | A/G |
| LNC-0052611 | LncRNA | SNP12 | A/T |
| Pto-COMT25 | 3′UTR | SNP13 | T/C |
| Pto-COMT25 | 3′UTR | SNP14 | A/T |
| Pto-COMT25 | 3′UTR | SNP15 | T/A |
| Pto-COMT25 | 3′UTR | SNP16 | T/C |
| Pto-COMT25 | 3′UTR | SNP17 | T/A |
| Pto-COMT25 | 3′UTR | SNP18 | A/C |
| Pto-COMT25 | 3′UTR | SNP19 | A/T |
| Pto-COMT25 | 3′UTR | SNP20 | T/C |
| Pto-COMT25 | 3′UTR | SNP21 | T/G |
| Pto-COMT25 | 3′UTR | SNP22 | C/T |
| Pto-COMT25 | 3′UTR | SNP23 | A/G |
| Pto-COMT25 | 3′UTR | SNP24 | A/C |
| Pto-COMT25 | 3′UTR | SNP25 | A/G |
| Pto-COMT25 | 3′UTR | SNP26 | G/C |
| Pto-COMT25 | 3′UTR | SNP27 | C/G |
| Pto-COMT25 | 3′UTR | SNP28 | T/C |
| Pto-COMT25 | 3′UTR | SNP29 | A/G |
| Pto-COMT25 | 3′UTR | SNP30 | C/T |
| Pto-COMT25 | 3′UTR | SNP31 | G/C |
| Pto-COMT25 | 3′UTR | SNP32 | T/C |
| Pto-COMT25 | 3′UTR | SNP33 | T/A |
| Pto-COMT25 | 3′UTR | SNP34 | C/T |
| Pto-COMT25 | 3′UTR | SNP35 | C/G |
| Pto-COMT25 | 3′UTR | SNP36 | T/A |
| Pto-COMT25 | 3′UTR | SNP37 | T/C |
| Pto-COMT25 | 3′UTR | SNP38 | T/C |
| Pto-COMT25 | 3′UTR | SNP39 | A/T |
| Pto-COMT25 | 3′UTR | SNP40 | T/C |
| Pto-COMT25 | 3′UTR | SNP41 | A/T |
| Pto-COMT25 | 3′UTR | SNP42 | T/C |
| Pto-COMT25 | 3′UTR | SNP43 | G/T |
| Pto-COMT25 | 3′UTR | SNP44 | A/G |
| Pto-COMT25 | 3′UTR | SNP45 | A/G |
| Pto-COMT25 | Coding region | SNP46 | A/G |
| Pto-COMT25 | Coding region | SNP47 | A/G |
| Pto-COMT25 | Coding region | SNP48 | C/T |
| Pto-COMT25 | Coding region | SNP49 | C/A |
| Pto-COMT25 | Coding region | SNP50 | A/T |
| Pto-COMT25 | Coding region | SNP51 | A/G |
| Pto-COMT25 | Coding region | SNP52 | A/G |
| Pto-COMT25 | Coding region | SNP53 | T/C |
| Pto-COMT25 | Coding region | SNP54 | C/T |
| Pto-COMT25 | Coding region | SNP55 | C/G |
| Pto-COMT25 | Coding region | SNP56 | T/C |
| Pto-COMT25 | Coding region | SNP57 | C/T |
| Pto-COMT25 | Coding region | SNP58 | T/C |
| Pto-COMT25 | Coding region | SNP59 | T/C |
| Pto-COMT25 | Coding region | SNP60 | G/T |
| Pto-COMT25 | Intron | SNP61 | T/C |
| Pto-COMT25 | Intron | SNP62 | A/G |
| Pto-COMT25 | Coding region | SNP63 | C/A |
| Pto-COMT25 | Coding region | SNP64 | G/A |
| Pto-COMT25 | Coding region | SNP65 | G/T |
| Pto-COMT25 | Coding region | SNP66 | C/T |
| Pto-COMT25 | Coding region | SNP67 | T/G |
| Pto-COMT25 | Coding region | SNP68 | C/T |
| Pto-COMT25 | Coding region | SNP69 | A/G |
| Pto-COMT25 | Coding region | SNP70 | G/A |
| Pto-COMT25 | Coding region | SNP71 | C/T |
| Pto-COMT25 | Coding region | SNP72 | G/A |
| Pto-COMT25 | Coding region | SNP73 | T/C |
| Pto-COMT25 | 5′UTR | SNP74 | G/A |
| Pto-COMT25 | 5′UTR | SNP75 | G/A |
| Pto-COMT25 | 5′UTR | SNP76 | T/C |
| Pto-COMT25 | 5′UTR | SNP77 | G/A |

Step 2: the mature xylems of 435 individuals in the natural population of P. tomentosa are collected, and the RNAs thereof are extracted respectively and transferred to the biotechnology company for transcriptome sequencing to obtain the population expression abundance data of genes expressed in the xylem of P. tomentosa, and the expression abundance of the candidate gene Pto-COMT25 in 435 individuals of the population is extracted.

Step 3: the DBH index of 435 individuals in the natural population of P. tomentosa is determined by using a growth trait measurement tool, and the phenotypic data of the index in the population is obtained.

Step 4: association analysis is performed using the SNPs within the lncRNA LNC-0052611 and the gene Pto-COMT25 and the population DBH index of P. tomentosa by using a mixed linear model in TASSEL v5.0 software, which is used for determining the SNP loci significantly associated with the DBH of P. tomentosa, where the determining condition includes: the SNP loci significantly associated with the plant target trait simultaneously includes the SNP loci in the plant candidate lncRNA and SNP loci in the plant candidate gene. The results show that SNP7 in the lncRNA LNC-0052611 and SNP45 and SNP61 in Pto-COMT25 are significantly associated with DBH trait (Table 2).

TABLE 2
Results of association analysis between SNPs in candidate
genetic factors and DBH trait in P. tomentosa

| Traits | SNP locus | SNP location | P value | Q value |
|---|---|---|---|---|
| DBH | SNP7 | LncRNA | 3.09 × 10⁻⁵ | 0.026 |
| | | LNC-0052611 | | |
| DBH | SNP45 | Pto-COMT25 | 3.31 × 10⁻⁴ | 0.032 |
| DBH | SNP61 | Pto-COMT25 | 8.27 × 10⁻⁴ | 0.055 |

Step S: association analysis is performed on the SNPs in lncRNA and the population expression levels of Pto-COMT25 by using the mixed linear model in the TASSEL v5.0 software, and the SNP loci significantly associated with Pto-COMT25 are screened, where the screening condition includes: the SNP loci within the plant candidate lncRNA are significantly associated with the expression level of the candidate gene. It is found that SNP2, SNP6, SNP7, and SNP11 in lncRNA LNC-0052611 are significantly associated with the expression level of Pto-COMT25 (Table 3), which indicates that LNC-0052611 can affect the expression of Pto-COMT25 to some extent.

TABLE 3
Results of association analysis between SNP in LNC-0052611
and the expression level of Pto-COMT25

| Traits | SNP locus | P value | Q value |
|---|---|---|---|
| Pto-COMT25 expression level | SNP2 | 3.52 × 10⁻⁵ | 0.022 |
| Pto-COMT25 expression level | SNP6 | 6.68 × 10⁻⁴ | 0.034 |
| Pto-COMT25 expression level | SNP7 | 1.62 × 10⁻³ | 0.052 |
| Pto-COMT25 expression level | SNP11 | 1.72 × 10⁻³ | 0.053 |

Step 6: the formula is calculated using the correlation coefficient, and the formula is as follows:

$$\frac{N \sum XY - \sum X \sum Y}{\sqrt{N \sum {X}^{2} - {( \sum X )}^{2}} \sqrt{N \sum {Y}^{2} - {( \sum Y )}^{2}}}$$

where X is the expression quantity data of the plant candidate gene in the detected tissue, and Y is the target trait population phenotypic data. The correlation coefficient between the expression quantity of Pto-COMT25 in the population and the DBH traits of the population is analyzed. The result shows that the correlation coefficient between the expression quantity and the DBH traits is r=0.553, which indicates that the expression level of Pto-COMT25 can affect the variation of DBH trait in P. tomentosa to some extent.

Step 7: the calculation results of steps (4) through (6) are comprehensively considered. The association results in step (4) showed that the SNP loci in lncRNA LNC-0052611 and Pto-COMT25 have a significant genetic effect on the variation of the DBH trait in P. tomentosa, which indicates that LNC-0052611 and Pto-COMT25 may affect the size of the DBH of P. tomentosa. The analysis results in step (5) indicated that LNC-0052611 may regulate the expression of Pto-COMT25. The research results in step (6) indicate that the expression level of Pto-COMT25 may affect the variation of the DBH trait of P. tomentosa to some extent. In view of the foregoing three points, an interaction relationship between lncRNA LNC-0052611 and the gene Pto-COMT25 exists, and their interaction affects the variation of the DBH trait in P. tomentosa.

It can be concluded from the above that an interaction relationship between P. tomentosa lncRNA LNC-0052611 and the gene Pto-COMT25 exists, and the interaction relationship affects the phenotypic variation of the DBH of P. tomentosa.

The embodiments described above are only descriptions of preferred embodiments of the present invention, and do not intended to limit the scope of the present invention. Various variations and modifications can be made to the technical solution of the present invention by those of ordinary skills in the art, without departing from the design and spirit of the present invention. The variations and modifications should all fall within the claimed scope defined by the claims of the present invention.

What is claimed is:

1. A method for identifying plant lncRNA and target gene interaction, comprising:
(1) obtaining population SNP genotype data of a plant candidate lncRNA and a plant candidate gene;
(2) obtaining population expression quantity data of the plant candidate gene in a studied tissue;
(3) performing phenotypic measurement on a plant target trait to obtain target trait population phenotypic data;
(4) performing association analysis on the population SNP genotype data in step (1) and the target trait population phenotypic data in step (3) to determine an SNP locus significantly associated with the plant target trait, wherein a determining condition for step (4) comprises: the SNP locus significantly associated with the plant target trait simultaneously comprises SNP loci in the plant candidate lncRNA and SNP loci in the plant candidate gene;
(5) performing association analysis on the population SNP genotype data in step (1) and the population expression quantity data in step (2) to determine an SNP locus associated with an expression level of the plant candidate gene, wherein a determining condition for step (5) comprises: the SNP locus of the plant candidate lncRNA is significantly associated with the expression level of the candidate gene;
(6) calculating a correlation coefficient r between the population expression data in step (2) and the target trait population phenotypic data in step (3) to determine a correlation therebetween, wherein a determining condition for step (6) comprises: the correlation coefficient r>0.5 or r<−0.5, with the formula for calculating the correlation coefficient r being as follows:
$$\frac{N \sum XY - \sum X \sum Y}{\sqrt{N \sum {X}^{2} - {( \sum X )}^{2}} \sqrt{N \sum {Y}^{2} - {( \sum Y )}^{2}}}$$
wherein X is the expression quantity data of the plant candidate gene in the studied tissue, and Y is the target trait population phenotypic data; and
(7) when the determining conditions in steps (4) through (6) are satisfied simultaneously, indicating that the plant candidate lncRNA and the plant candidate gene have an interaction relationship, and together affect a phenotypic variation of the plant target trait.

2. The method of claim 1, wherein the plant candidate lncRNA and the plant candidate gene in step (1) are expressed in the same tissue of a plant.

3. The method of claim 1, wherein the population SNP genotype data in step (1) is obtained based on plant whole genome re-sequencing data.

4. The method of claim 3, wherein the method for obtaining the population SNP genotype data in step (1) comprises:
performing whole genome sequencing on each individual in a used natural population to respectively obtain genomic sequences;
performing sequence alignment on the genomic sequences to obtain whole genome genotype SNP data; and
performing alignment on the plant candidate lncRNA and the plant candidate gene and a reference genome, and combining the whole genome genotype SNP data to obtain the population SNP genotype data.

5. The method of claim 1, wherein a frequency of the population SNP genotype data of the plant candidate lncRNA and the plant candidate gene in step (1) is greater than 10%.

6. The method of claim 1, wherein software used for the association analysis in step (4) is TASSEL v5.0.

7. The method of claim 6, wherein a model used for the association analysis is a mixed linear model.

8. The method of claim 7, wherein the association analysis method comprises:
obtaining a significance level P value of each SNP locus associated with a phenotype by using the software TASSEL v5.0;
performing FDR multiple tests on the P value by using Q-value software to obtain a Q value; and
screening SNP loci with P≤0.01 and Q≤0.1 as SNP loci significantly associated with the plant target traits.

9. The method according to claim 1, wherein the method for obtaining the population SNP genotype data in step (1) comprises:
performing whole genome sequencing on each individual in a used natural population to respectively obtain genomic sequences;
performing sequence alignment on the genomic sequences to obtain whole genome genotype SNP data; and
performing alignment on the plant candidate lncRNA and the plant candidate gene and a reference genome, and combining the whole genome genotype SNP data to obtain the population SNP genotype data.

10. The method of claim 1, wherein a model used for the association analysis is a mixed linear model.

11. The method of claim 10, wherein the association analysis method comprises:
obtaining a significance level P value of each SNP locus associated with a phenotype by using the software TASSEL v5.0;
performing FDR multiple tests on the P value by using Q-value software to obtain a Q value; and
screening SNP loci with P≤0.01 and Q≤0.1 as SNP loci significantly associated with the plant target traits.
